(19) United States
(12) Patent Application Publication
(10) Pub. No.: US 2022/0003778 A1
(43) Pub. Date: Jan. 6, 2022

(54) METHOD FOR ANALYZING MICROORGANISM

(71) Applicant: SHIMADZU CORPORATION, Kyoto-shi (JP)

(72) Inventor: Yuko FUKUYAMA, Kyoto-shi (JP)

(73) Assignee: SHIMADZU CORPORATION, Kyoto-shi (JP)

(21) Appl. No.: 17/232,236

(22) Filed: Apr. 16, 2021

(30) Foreign Application Priority Data
Apr. 23, 2020 (JP) 2020-076954

(51) Int. Cl. G01N 33/68; G01N 33/50; H01J 49/00

(52) U.S. Cl. CPC G01N 33/6851; H01J 49/0036; G01N 33/5005

(57) ABSTRACT

A method for analyzing a microorganism according to the present invention includes: a step of reading, from a mass spectrum obtained by a mass spectrometric analysis of a sample containing a microorganism, a mass-to-charge ratio or ratios m/z of a peak or peaks originating from 1-10 kinds of proteins selected from the group, consisting of 10 kinds of proteins of gns, YaiA, YibT, PPI, L25, L21, S8, L17, L15 and S7 as well as any combination of the 10 kinds of proteins; and an identification step configured to determine, based on the read mass-to-charge ratio or ratios, m/z, of the peak or peaks, whether or not the microorganism contained in the sample contains at least one of the following six serotypes of Salmonella bacteria: Abaetetuba, Anatum, Newport, Poona, Tallahassee and Vellore.

### TECHNICAL FIELD

The present invention relates to a method for analyzing a microorganism.

### BACKGROUND ART

A matrix assisted laser desorption/ionization method, which is one type of ionization method in mass spectrometry, is an ionization method for analyzing a substance that barely absorbs laser light, or a substance that is likely to be damaged by laser light, such as proteins. A matrix substance, which considerably absorbs laser light and is thereby easily ionized, is mixed with a substance to be analyzed, and the mixture is irradiated with laser light to ionize the substance to be analyzed. Typically, the matrix substance is prepared in the form of a solution and is mixed with the substance to be analyzed. The solvent in the solution is subsequently vaporized to obtain a dried matrix in the form of a crystal containing the substance to be analyzed. Then, the mixture is irradiated with laser light, whereby the matrix substance absorbs the energy of the laser light and becomes rapidly heated to be ultimately vaporized. The substance to be analyzed is also vaporized with the matrix substance. Through this process, the substance to be analyzed becomes ionized.

A mass spectrometer using such a MALDI method (MALDI-MS) can analyze a high-molecular compound, such as a protein, without causing a significant dissociation of the compound, and is also suitable for microanalysis. Therefore, this type of mass spectrometer has been widely used in the area of life science. One application of the MALDI-MS in the area of life science is the identification of microorganisms using MALDI-MS. This is a method in which a microorganism is identified based on a mass spectrum pattern obtained using a test microorganism. Since this method can provide analysis results within a short period of time, the identification of a microorganism can be conveniently and speedily performed.

For example, a representative causative organism of food poisoning is Salmonella, which is a group of rod-shaped gram-negative facultatively anaerobic bacteria of the family Enterobacteriaceae. There are three species belonging to Salmonella: Salmonella (which is hereinafter abbreviated as “S.”) enterica, S. bongori and S. subterranea. S. enterica is further divided into six subspecies. Many of the pathogenic Salmonella causative of food poisoning belong to S. enterica subsp. enterica. This subspecies is further divided into a large number of serotypes. Determining the species, subspecies and serotypes of Salmonella bacteria is important for elucidating the infection route of food poisoning and preventing the infection. Therefore, in recent years, attempts have been made to identify Salmonella bacteria using MALDI-MS.

### CITATION LIST

### Non Patent Literature

Non Patent Literature 1: Applied Microbiology and Biotechnology, Vol. 101, issue 23-24, pp. 8557-8569, 2017

### SUMMARY OF INVENTION

### Technical Problem

Determination of the species, subspecies and serotypes of Salmonella bacteria by MALDI-MS is achieved by detecting a biomarker peak, i.e., a peak whose position (mass-to-charge ratio; m/z value) and height (peak intensity; mV value) in the mass spectrum vary among bacterial bodies of different species, subspecies and serotypes. For the identification of microorganisms including bacteria, a protein peak is often used as a biomarker peak (see Non Patent Literature 1).

For many proteins in closely related microorganisms, it is normally the case that a peak originating from the same protein appears at the same mass-to-charge ratio or within a narrow range of mass-to-charge ratios. Therefore, for an accurate identification of Salmonella bacteria at the level of serotype, it is not enough to select a peak originating from one protein as a biomarker peak; it is necessary to select peaks originating from a plurality of appropriate kinds of proteins for the serotype concerned. Additionally, there are only a limited number of serotypes which are known to be identifiable through the use of biomarker peaks. Identifying an even greater number of serotypes has been desired.

The problem to be solved by the present invention is to enable the identification of even more serotypes of Salmonella bacteria with the highest possible level of accuracy in a method for analyzing a microorganism using MALDI-MS.

### Solution to Problem

A method for analyzing a microorganism according to the present invention developed for solving the previously described problem includes:

a step of reading, from a mass spectrum obtained by a mass spectrometric analysis of a sample containing a microorganism, a mass-to-charge ratio or ratios m/z of a peak or peaks originating from 1-10 kinds of proteins selected from the group, consisting of 10 kinds of proteins of gns, YaiA, YibT, PPI, L25, L21, S8, L17, L15 and S7 as well as any combination of the 10 kinds of proteins; and

an identification step configured to determine, based on the read mass-to-charge ratio or ratios, m/z, of the peak or peaks, whether or not the sample contains at least one of the following six serotypes of Salmonella bacteria: Abaetetuba, Anatum, Newport, Poona, Tallahassee and Vellore.

### Advantageous Effects of Invention

According to the present invention, it is possible to determine that a sample contains bacteria whose serotype is one of the six predetermined serotypes of Salmonella that have conventionally been difficult to be distinguished from other serotypes of Salmonella.

### BRIEF DESCRIPTION OF DRAWINGS

FIG. 1 is a schematic overall configuration diagram of a microorganism-analyzing system used for a method for analyzing a microorganism according to the present invention.

FIG. 2 is a flowchart showing one example of the procedure of the method for analyzing a microorganism.

FIG. 3 shows the mass-to-charge ratios of peaks originating from 10 kinds of proteins, obtained for samples containing each of the bacterial bodies of six serotypes of Salmonella.

FIG. 4 shows the mass-to-charge ratios of peaks originating from 10 kinds of proteins, obtained for samples containing each of the bacterial bodies of 22 serotypes of Salmonella.

FIG. 5 shows mass spectra of proteins YaiA and L25 in three serotypes of Salmonella bacteria belonging to the first group.

### DESCRIPTION OF EMBODIMENTS

The method for analyzing a microorganism according to the present invention includes:

a step of reading, from a mass spectrum obtained by a mass spectrometric analysis of a sample containing a microorganism, a mass-to-charge ratio or ratios m/z of a peak or peaks originating from 1-10 kinds of proteins selected from the group, consisting of 10 kinds of proteins of gns, YaiA, YibT, PPI, L25, L21, S8, L17, L15 and S7 as well as any combination of the 10 kinds of proteins; and

an identification step configured to determine, based on the read mass-to-charge ratio or ratios, m/z, of the peak or peaks, whether or not the sample contains at least one of the following six serotypes of Salmonella bacteria: Abaetetuba, Anatum, Newport, Poona, Tallahassee and Vellore.

In Non Patent Literature 1, 12 kinds of proteins (gns, YaiA, YibT, PPI, L25, L21, S8, L17, L15, S7, YciF, SodA) are reported to be useful as biomarkers for identifying 22 serotypes of Salmonella. The present invention has been conceived from the new finding that whether or not a sample contains any one of the six serotypes of bacterial bodies can be determined by using, as biomarkers, 10 kinds of proteins (gns, YaiA, YibT, PPI, L25, L21, S8, L17, L15 and S7) included in the 12 kinds of proteins.

It has been known that, if at least one of the six serotypes of bacterial bodies is contained in a sample, at least one of the peaks present in a mass spectrum obtained for that sample is a peak originating from one of the 10 kinds of proteins. Each of the 10 kinds of proteins has a known mass-to-charge-ratio range within which a peak originating from that protein should appear. Accordingly, whether or not a peak is present is determined for each of the mass-to-charge-ratio ranges corresponding to the 10 kinds of proteins on the mass spectrum obtained for the sample. If a peak is present within at least one of the mass-to-charge-ratio ranges corresponding to the 10 kinds of proteins, it is possible that the sample contains at least one of the six serotypes of bacterial bodies. If no such peak is present, it is possible to determine that the sample contains none of the six serotypes of fungus bodies.

Another method for analyzing a microorganism according to the present invention includes:

a step of reading, from a mass spectrum obtained by a mass spectrometric analysis of a sample containing a microorganism, a mass-to-charge ratio or ratios m/z of a peak or peaks originating from 1-10 kinds of proteins selected from the group, consisting of 10 kinds of proteins of gns, YaiA, YibT, PPI, L25, L21, S8, L17, L15 and S7 as well as any combination of the 10 kinds of proteins; and

an identification step configured to determine, based on the read mass-to-charge ratio or ratios, m/z, of the peak or peaks, whether or not the sample contains at least one of the following six serotypes of Salmonella bacteria: Abaetetuba, Anatum, Newport, Poona, Tallahassee and Vellore, or whether or not the sample contains at least one of the following 22 serotypes of Salmonella bacteria: Enteritidis, Typhimurium, Minnesota, Infantis, Brandenburg, Rissen, Schwarzengrund, Montevideo, Mbandaka, Orion, Pullorum_Gallinarum, Abony, Choleraesuis, Saintpaul, Braenderup, Pakistan, Thompson, Altona, Istanbul, Manhattan, Senftenberg, and Amsterdam.

It has been known that, if a sample contains at least one of the six serotypes of bacterial bodies, or at least one of the 22 serotypes of bacterial bodies, at least one of the peaks present in a mass spectrum obtained for the sample is a peak originating from one of the 10 kinds of proteins. Each of the 10 kinds of proteins has a known mass-to-charge-ratio range within which a peak originating from that protein should appear. Accordingly, whether or not a peak is present is initially determined for each of the mass-to-charge-ratio ranges corresponding to the 10 kinds of proteins on the mass spectrum obtained for the sample.

If a peak is present within at least one of the mass-to-charge-ratio ranges corresponding to the 10 kinds of proteins, the value of the mass-to-charge ratio m/z of that peak is read. After one or more values of the mass-to-charge ratio m/z have been read, it is possible to determine that the sample contains at least one of the six serotypes of bacterial bodies, or at least one of the 22 serotypes of bacterial bodies, based on that value of the mass-to-charge ratio m/z, or based on the combination of a plurality of values of the mass-to-charge ratio m/z.

In the previously described method for analyzing a microorganism, if it has been determined that the sample contains at least one of the six serotypes of Salmonella bacteria, it is possible to determine which of the six serotypes is the serotype of Salmonella bacteria contained in the sample, based on the mass-to-charge ratio or ratios m/z of a peak or peaks originating from 1-10 kinds of proteins selected from the group, consisting of the 10 kinds of proteins and any combination of the 10 kinds of proteins.

Another method for analyzing a microorganism according to the present invention includes:

a step of reading, from a mass spectrum obtained by a mass spectrometric analysis of a sample containing a microorganism, a mass-to-charge ratio or ratios m/z of a peak or peaks originating from 1-10 kinds of proteins selected from the group, consisting of 10 kinds of proteins of gns, YaiA, YibT, PPI, L25, L21, S8, L17, L15 and S7 as well as any combination of the 10 kinds of proteins; and

a step of determining which of the six aforementioned serotypes is contained in the sample, based on the read mass-to-charge ratio or ratios, m/z, of the peak or peaks.

As a mass spectrometer to be used for the method for analyzing a microorganism according to the present invention, a mass spectrometer using a matrix assisted laser desorption/ionization (MALDI) method (MALDI-MS) is preferable. As the MALDI-MS, a MALDI time-of-flight mass spectrometer (MALDI-TOFMS) can preferably be used. Since the MALDI-MS has an extremely wide range of measurable mass-to-charge ratios, a mass spectrum suited for an analysis of high-mass molecules, such as the proteins which are constituents of microorganisms, can be acquired.

Next, one embodiment of the microorganism-analyzing system to be used for the method for analyzing a microorganism according to the present invention is described.

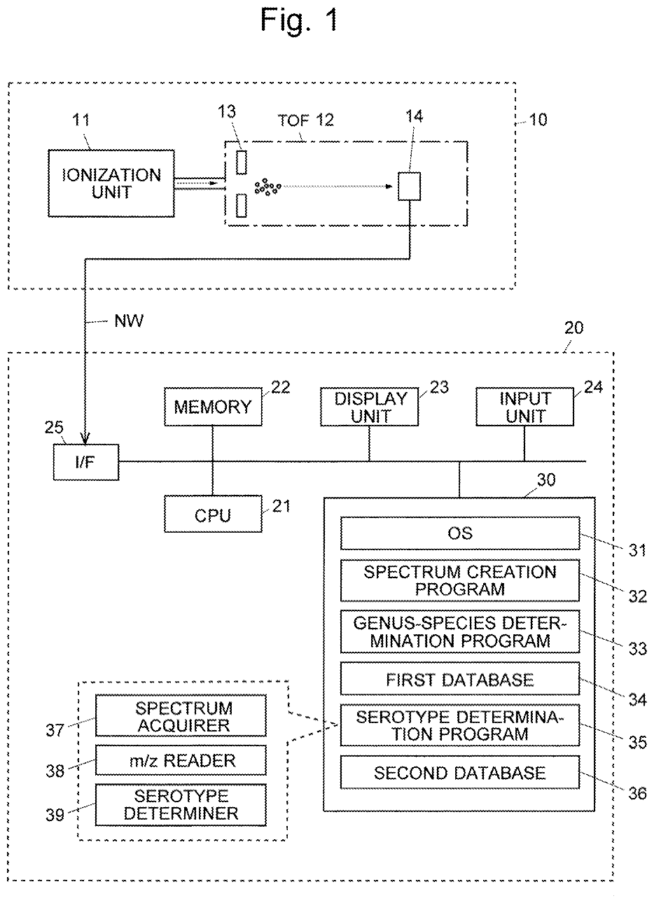

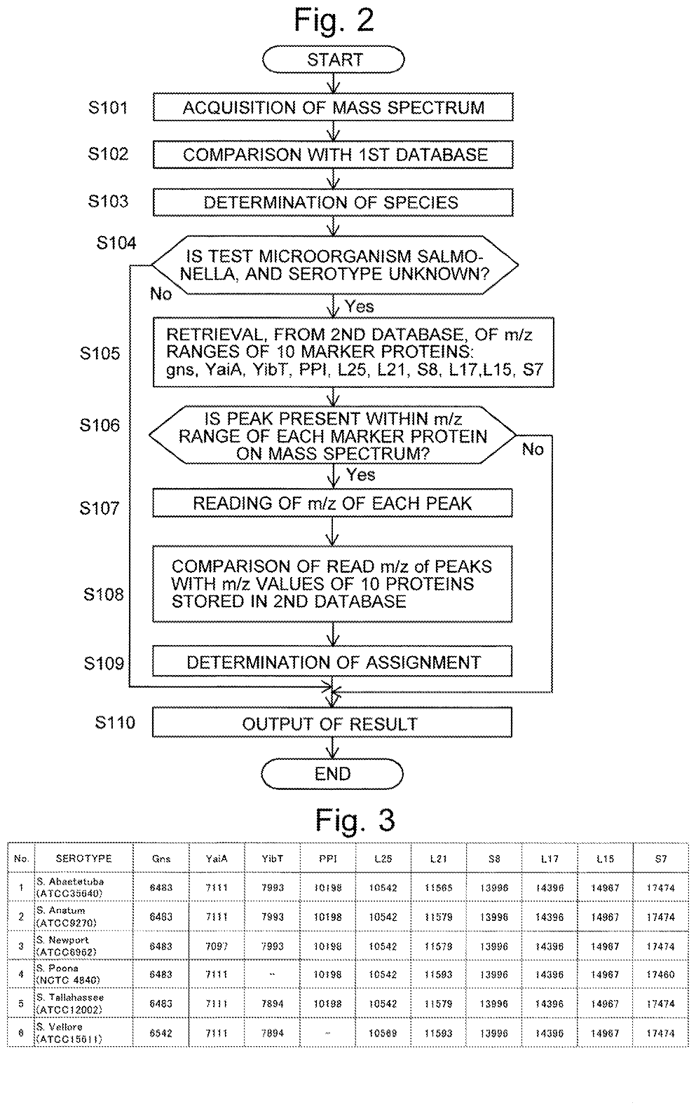

FIG. 1 shows a schematic overall configuration of the microorganism-analyzing system. This system is roughly divided into a mass spectrometry unit 10 and a microorganism identification unit 20. The mass spectrometry unit 10 includes an ionization unit 11 configured to ionize molecules or atoms in a sample by matrix assisted laser desorption/ionization (MALDI) and a time-of-flight mass separator (TOF) 12 configured to separate various ions, ejected from the ionization unit 11, according to their mass-to-charge ratios.

The TOF 12 includes an extraction electrode 13 configured to extract ions from the ionization unit 11 and guide them into an ion flight space within the TOF 12, and a detector 14 configured to detect ions which have been mass-separated within the ion flight space.

The microorganism identification unit 20 is actually a workstation, personal computer or other types of computers, in which a central processing unit (CPU) 21, memory 22, display unit 23 (e.g., a liquid crystal display), input unit 24 (e.g., a keyboard and mouse), and storage unit 30 consisting of a large-capacity storage (e.g., a hard disk drive or solid state drive) are connected to each other. Stored in the storage unit 30 are an operating system (OS) 31, spectrum creation program 32, species determination program 33 and serotype determination program 35 (a program according to the present invention), as well as a first database 34 and second database 36. The microorganism identification unit 20 further includes an interface (I/F) 25 for controlling a direct connection to an external device as well as a connection with an external device through a local area network (LAN) or other types of networks. Through this interface 25, the microorganism identification unit 20 is connected with the mass spectrometry unit 10 by a network cable NW (or wireless LAN).

In FIG. 1, a spectrum acquirer 37, m/z reader 38 and serotype identifier 39 are shown, being linked to the serotype determination program 35. Each of those components is basically a functional means implemented at the software level by the CPU 21 executing the serotype determination program 35. The serotype determination program 35 does not need to be an independent program. There is no specific limitation on its form; for example, it may be a built-in function of the species determination program 33 or that of a program for controlling the mass spectrometry unit 10. As the species determination program 33, for example, a program configured to identify microorganisms by a conventional fingerprinting method may be used.

In the configuration in FIG. 1, the spectrum creation program 32, species determination program 33, serotype determination program 35, first database 34 and second database 36 are installed on a terminal device to be operated by users. Those components may be at least partially, or even entirely, installed on a separate device connected with the aforementioned terminal device via a computer network, with the separate device configured to perform the processing by those programs and/or access to those databases according to commands from the terminal device.

The first database 34 in the storage unit 30 holds a large number of mass lists related to known microorganisms. The mass list is a list of the mass-to-charge ratios of ions to be detected in a mass spectrometric analysis of a specific kind of microorganic cell. Along with the information of the mass-to-charge ratios, the list additionally includes at least the information of the classifications (family, genus, species, etc.) to which the microorganic cell belongs (classification information). Those mass lists should preferably be prepared based on actual measurement data obtained beforehand by actually performing mass spectrometric analyses of various kinds of microorganic cells using the same method for ionization and mass separation as used in the mass spectrometry unit 10.

When the mass lists are to be prepared from the actual measurement data, the peaks which appear within a predetermined mass-to-charge-ratio range are initially extracted from mass spectra obtained as the actual measurement data. Peaks which mainly originate from proteins can be extracted by setting the aforementioned mass-to-charge-ratio range at approximately 4000-30000, while unwanted peaks (noise) can be excluded by extracting each peak whose height (relative intensity) is equal to or higher than a predetermined threshold. A list of the mass-to-charge ratios (m/z) of the extracted peaks is created for each kind of cell and recorded in the first database 34 along with the aforementioned classification information and other related information. In order to reduce the variation in generic expression due to the culture conditions, the microorganic cells to be used for collecting the actual measurement data should preferably be cultured under previously normalized conditions.

The second database 36 in the storage unit 30 holds information concerning marker proteins for identifying, known kinds of microorganisms, by their serotypes which are classifications lower than the species. The information concerning the marker proteins includes at least the information of the mass-to-charge ratios (m/z) of the marker proteins in the known kinds of microorganisms. The second database 36 may also hold information concerning marker proteins for identifying known kinds of microorganisms by another sub-classification (e.g., subspecies, pathotype or strain) other than the serotype, or by other criteria.

The second database 36 in the present embodiment holds mass-to-charge-ratio ranges which respectively correspond to at least 10 kinds of proteins, i.e., gns, YaiA, YibT, PPI, L25, L21, S8, L17, L15 and S7, as well as the value of one or more mass-to-charge ratios within those mass-to-charge-ratio ranges, as information concerning marker proteins for determining whether or not a test microorganism is a Salmonella bacterium belonging to the first group shown below, or whether or not it is a Salmonella bacterium belonging to the second group shown below, or for determining which of the first group of Salmonella bacteria is the test microorganism, or which of the second group of Salmonella bacteria is the test microorganism.

### <First Group>

(1) S. Abaetetuba, ATCC35640
(2) S. Anatum, ATCC9270
(3) S. Newport, ATCC6962
(4) S. Poona, NCTC 4840
(5) S. Tallahassee, ATCC12002
(6) S. Vellore, ATCC15611

### <Second Group>

(1) S. Enteritidis, GTC08914
(2) S. Typhimurium, NBRC13245
(3) S. Minnesota, NBRC15182
(4-1) S. Infantis, ATCCBAA-1675
(4-1) S. Infantis, jfrlSe1402-1
(5) S. Brandenburg, jfrlSe1402-3
(6) S. Rissen, jfrlSe1402-6
(7) S. Schwarzengrund, HyogoSO12004
(8) S. Montevideo, jfr1Se1409-6
(9-1) S. Mbandaka, jfrlSe1402-13
(9-2) S. Mbandaka, jfrlSe1402-14
(10) S. Orion, jfrlSe1402-7
(11) S. Pullorum_Gallinarum, NBRC3163
(12) S. Abony, NBRC100797
(13) S. Choleraesuis, NBRC105684
(14) S. Saintpaul, ATCC9712
(15) S. Braenderup, GTC09492
(16) S. Pakistan, GTC09493
(17) S. Thompson, ATCC BAA-1738
(18) S. Altona, jfrlSe1409-1
(19) S. Istanbul, jfrlSe1409-2
(20) S. Manhattan, HyogoSO11001
(21) S. Senftenberg, jfrlSe1409-3
(22) S. Amsterdam, jfrlSe1409-21

The members of the first group are six kinds of Salmonella bacteria classified into six serotypes. The members of the second group are 24 kinds of Salmonella bacteria classified into 22 serotypes. Among the 22 serotypes, the serotypes Infantis and Mbandaka each include two kinds of Salmonella bacteria. Salmonella bacteria classified into the same serotype are denoted by the same number, with sub-numbers (branch) 1 and 2.

The values of the mass-to-charge ratios of the marker proteins stored in the second database 36 should preferably be selected by comparing a calculation mass determined by translating the base sequence of each marker protein into an amino-acid sequence, and a mass-to-charge ratio detected by an actual measurement. The base sequences of the marker proteins may be determined by sequencing method, or they may be retrieved from public databases, e.g., a database at NCBI (National Center for Biotechnology Information). For the determination of the calculation mass from the amino-acid sequence, the cutting of the N-terminal methionine residue should preferably be taken into account as a post-translational modification. Specifically, if the second amino-acid residue to the last is Gly, Ala, Ser, Pro, Val, Thr or Cys, the theoretical value should be calculated on the assumption that the N-terminal methionine will be cut. Additionally, since the molecule to be actually observed with a MALDI-TOF MS is in a protonated form, the addition of the proton should also preferably be taken into account in determining the calculation mass (i.e., a theoretical value of the mass-to-charge ratio of an ion to be obtained in an analysis of a protein with a MALDI-TOF MS).

Next, a procedure of the analysis of the serotype of Salmonella bacteria using the previously described microorganism-analyzing system is described with reference to the flowchart.

Initially, the user prepares a sample containing the constituents of a test microorganism, sets the sample in the mass spectrometry unit 10, and operates the same unit to perform the mass spectrometric analysis. The sample may be a cell extract, or cell constituents (e.g., ribosomal proteins) collected from the cell extract and purified. Bacterial bodies or cell suspension may also be used as they are.

The spectrum creation program 32 receives detection signals from the detector 14 via the interface 25, and creates a mass spectrum for the test microorganism based on the detection signals (Step 101).

Next, the species determination program 33 compares the mass spectrum of the test microorganism with the mass lists of known microorganisms recorded in the first database 34, and extracts a mass list of a known microorganism having a similar pattern of mass-to-charge ratios to that of the mass spectrum of the test microorganism, such as a mass list including a considerable number of peaks which coincide with those of the mass spectrum of the test microorganism within a predetermined margin of error (Step 102). The species determination program 33 subsequently searches the first database 34 for the classification information related to the mass list extracted in Step 102, to determine the organism species to which the known microorganism corresponding to the mass list belongs (Step 103). If the organism species is not Salmonella (“No” in Step 104), the organism species is displayed on the display unit 23 as the organism species of the test microorganism (Step 110), and the analytical processing is completed. If the organism species is Salmonella (“Yes” in Step 104), the analysis proceeds to the processing by the serotype determination program 35. In the case where it has been previously determined by another method that the sample contains Salmonella bacteria, and the analysis can directly proceed to the serotype determination program 35 without using the species determination program using the mass spectrum.

In the serotype determination program 35, the serotype determiner 39 initially retrieves the mass-to-charge-ratio ranges of the 10 kinds of proteins gns YaiA, YibT, PPI, L25, L21, S8, L17, L15 and S7 which are biomarker proteins (i.e., 10 mass-to-charge-ratio ranges) from the second database 36 (Step 105). Subsequently, the spectrum acquirer 37 obtains the mass spectrum of the test microorganism prepared in Step 101. Then, for each of the marker proteins, the m/z reader 38 determines whether or not a peak is present on the mass spectrum within the mass-to-charge-ratio range related to the marker protein in the second database 36 (Step 106).

If no peak is present within any of the 10 mass-to-charge-ratio ranges stored in the second database 36 (“No” in Step 106), the Salmonella bacteria contained in the sample are displayed on the display unit 23 as a serotype which belongs to neither the first group nor the second group (Step 110), and the identification processing is completed.

On the other hand, if a peak is present within at least one of the 10 mass-to-charge-ratio ranges (“Yes” in Step 106), that peak is selected as a peak corresponding to the marker protein related to the mass-to-charge-ratio range within which that peak has been located, and the mass-to-charge ratio of that peak is read (Step 107). Subsequently, the serotype determiner 39 compares the read mass-to-charge ratio with each of the mass-to-charge-ratio values of the 10 marker proteins read from the second database 36, and determines whether or not the two values coincide with each other within a predetermined allowable error range (Step 108). If a plurality of values are stored in the second database 36 as the mass-to-charge ratios included within one mass-to-charge-ratio range, the serotype determiner 39 additionally determines which of those values coincides with the read value. As a result, if a value coinciding with the read mass-to-charge-ratio value has been found among the mass-to-charge ratios stored in the second database 36, the serotype determiner 39 determines, based on that result, that the test microorganism is a serotype belonging to the first group or a serotype belonging to the second group, or determines which of the serotypes belonging to the first and second groups corresponds to the test microorganism (Step 109). The determination result is displayed on the display unit 23 as the identification result (Step 110).

In the case where it has been previously determined by another method that the Salmonella bacteria contained in the sample belong to either the first group or second group, the determination on the presence or absence of a peak in Step 106 can be omitted, and the analysis can directly proceed to Step 107.

### EXAMPLE

Hereinafter described is an experiment conducted to prove the effect of the method for analyzing a microorganism according to the present invention. It should be noted that the following descriptions are merely illustrative and do not limit the present invention.

### 1. Culturing of Salmonella Bacteria

A total of 30 kinds of Salmonella bacteria, i.e., the six aforementioned kinds of Salmonella bacteria belonging to the first group and the 24 aforementioned kinds of Salmonella bacteria belonging to the second group, were cultured at 37 degrees Celsius for 20 hours using LB agar.

### 2. Preparation of Matrix Solutions

The following two kinds of matrix solutions were prepared.
(2-1) Sinapine acid (SA) as the matrix substance was dissolved in ethanol to obtain a matrix solution (saturated solution) with an SA content of 25 mg/mL. This matrix solution is hereinafter called “SA-1”.
(2-2) SA, methylene diphosphonate (MDPNA) and decyl-β-D-maltopyranoside (DMP) as a surfactant were dissolved in an aqueous solution with an acetonitrile (ACN) content of 50% and trifluoroacetic acid (TFA) content of 0.6% to obtain a matrix solution with an SA content of 25 mg/mL, MDPNA content of 1%, and DMP content of 1 mM. This matrix solution is hereinafter called “SA-2”.

The SA used for the matrix solutions SA-1 and SA-2 was a product of FUJIFILM Wako Pure Chemical Corporation. The MDPNA and DMP were products of Sigma-Aldrich Japan LLC.

### 3. Preparation of Matrix-Microorganism Suspension

(3-1) From each of the 30 kinds of Salmonella bacteria cultured on LB agar, an approximately 1 mg of sample was collected using a microbalance and put in a tube. The matrix solution SA-2 was added to the sample in the tube to obtain a solution with a bacteria concentration of 1 mg/0.075 mL (approximately 1×10⁷ CFU/4), and this solution was suspended with a needle.
(3-2) Ultrasonic vibrations were applied to the tube for one minute. The obtained suspension was centrifuged (at 12000 rpm for 5 minutes) to obtain a centrifugation supernatant.
   4. Analysis with MALDI-MS
(4-1) The matrix solution SA-1 was dropped into the wells on a MALDI sample plate at 0.54 per one well (precoating).
(4-2) Subsequently, the centrifugation supernatant was dropped into the wells precoated with the matrix solution SA-1, at 14 per one well, and was let to naturally dry.
(4-3) The MALDI sample plate obtained in (4-2) was set in a MALDI-MS (AXIMA Performance, manufactured by Shimadzu Corporation), and the measurement was performed in a linear mode (positive ion mode). All measurement data were acquired by a raster analysis. Raster analysis is an automatic measurement function provided in the previously mentioned mass spectrometer. In this technique, the sample in each well of the sample plate is irradiated with a predetermined number of laser shots at a predetermined number of points, to acquire mass spectrum data.

### 5. Results

A self-calibration of Salmonella bacteria was applied to the measurement data, and the mass-to-charge ratios m/z of the peaks originating from the 10 kinds of proteins (gns, YaiA, YibT, PPI, L25, L21, S8, L17, L15 and S7) were read from the obtained mass spectra. The self-calibration is a calibration process in which some peaks that have already been assigned to specific Salmonella bacteria are used as internal standards.

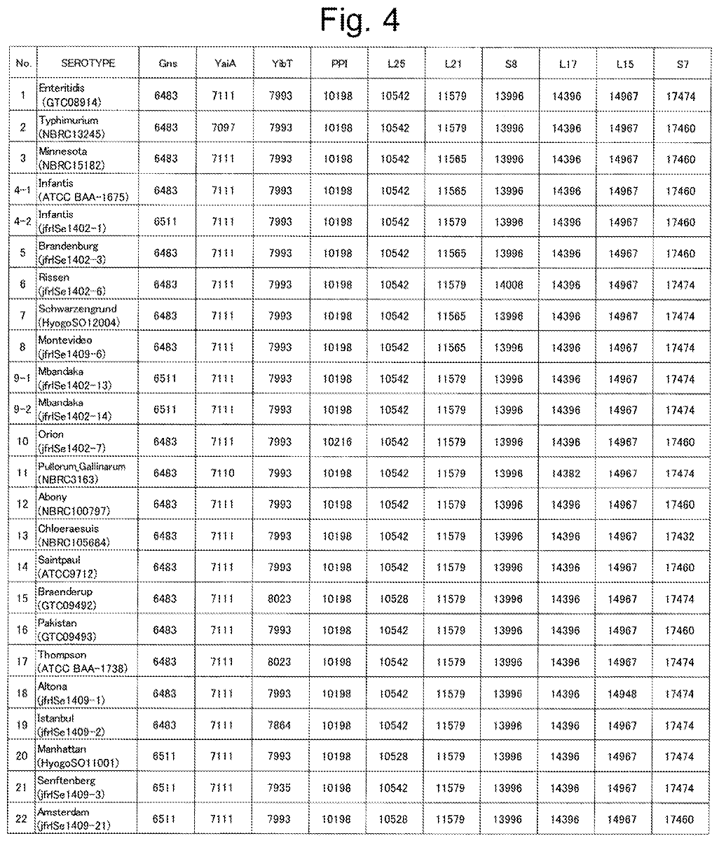

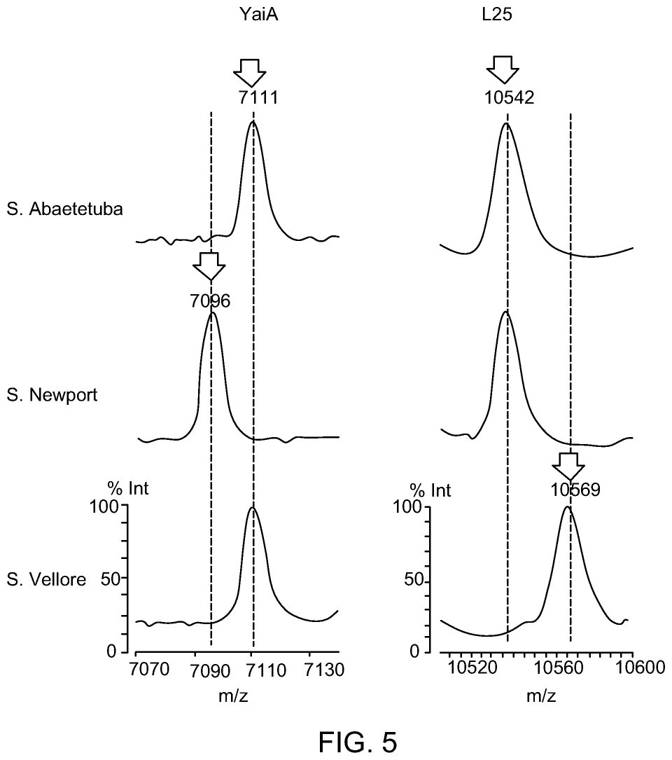

The results are shown in FIGS. 3-5. FIG. 3 is the result obtained for the six kinds of Salmonella bacteria belonging to the first group. FIG. 4 is the result obtained for the 24 kinds of Salmonella bacteria belonging to the second group. FIG. 5 shows mass spectra of proteins YaiA and L25 in Abaetetuba, Newport and Vellore which are three serotypes of Salmonella bacteria belonging to the first group. The hyphen characters (“-”) in FIG. 3 indicate that no peak was detected, at least this time.

As can be seen in FIGS. 3 and 4, some of the values of the mass-to-charge ratios of the peaks originating from the 10 kinds of proteins are common to a plurality of serotypes. However, an appropriate selection of one value or a combination of two or more values from of the mass-to-charge ratios of the peaks originating from the 10 kinds of proteins makes it possible to identify one of the serotypes in the first and second groups or narrow down the candidates. If the combination of the mass-to-charge ratios are common to a plurality of serotypes, the candidates can be narrowed down to those serotypes.

Specifically, in the case where it is previously known that the test microorganism is a Salmonella bacterium whose serotype belongs to either the first group or second group, if the value of the mass-to-charge ratio corresponding to the marker protein Gns is 6542, or if the value of the mass-to-charge ratio corresponding to L25 is 10569, the serotype is identified as Vellore of the first group. If the value of the mass-to-charge ratio corresponding to the marker protein YaiA is 7097, the serotype is identified as either Newport of the first group or Typhimurium of the second group.

In the case where it is previously known that the test microorganism is a Salmonella bacterium whose serotype belongs to either the first group or second group, if the value of the mass-to-charge ratio corresponding to the marker protein YibT is 7894, the serotype is identified as either Tallahassee or Vellore of the first group. If the value of the mass-to-charge ratio corresponding to L21 is 11593, the serotype is identified as either Poona or Vellore of the first group.

In the case where it is previously known that the test microorganism is a Salmonella bacterium whose serotype belongs to either the first group or second group, if the value of the mass-to-charge ratio corresponding to the marker protein YaiA is 7110, the serotype is identified as Pullorum_Gallinarum of the second group. If the value of the mass-to-charge ratio corresponding to PPI is 10216, the serotype is identified as Orion of the second group. If the value of the mass-to-charge ratio corresponding to L15 is 14948, the serotype is identified as Altona of the second group.

In the case where it is previously known that the test microorganism is a Salmonella bacterium whose serotype belongs to either the first group or second group, if the value of the mass-to-charge ratio corresponding to the marker protein YibT is 8023, the serotype is identified as Braenderup or Thompson of the second group. If the value of the mass-to-charge ratio corresponding to L25 is 10528, the serotype is identified as Braenderup, Manhattan or Amsterdam of the second group. If the value of the mass-to-charge ratio corresponding to the marker protein Gns is 6511, the serotype is identified as Infantis, Mbandaka, Manhattan, Senftenberg or Amsterdam of the second group.

In the case where it is previously known that the test microorganism is a Salmonella bacterium whose serotype belongs to the first group, if the mass-to-charge ratio corresponding to the marker protein L21 is 11565 and the mass-to-charge ratio corresponding to S7 is 17474, or if the combination of the mass-to-charge ratios of the 10 kinds of marker proteins is the combination of the numerical values shown in the row “No. 1” in FIG. 3, the serotype is identified as Abaetetuba. In the case where the test microorganism is a Salmonella bacterium with no information about its serotype, if the combination of the mass-to-charge ratios of the 10 kinds of marker proteins is the combination of the numerical values shown in the row “No. 1” in FIG. 3, it is confirmed that the serotype is possibly Abaetetuba (the candidates can be narrowed down).

In the case where it is previously known that the test microorganism is a Salmonella bacterium whose serotype belongs to the first group, if the mass-to-charge ratio corresponding to the marker protein L21 is 11579, the mass-to-charge ratio corresponding to YaiA is 7111 and the mass-to-charge ratio corresponding to YibT is 7993, or if the combination of the mass-to-charge ratios of the 10 kinds of marker proteins is the combination of the numerical values shown in the row “No. 2” in FIG. 3, the serotype is identified as Anatum. In the case where the test microorganism is a Salmonella bacterium with no information about its serotype, if the combination of the mass-to-charge ratios of the 10 kinds of marker proteins is the combination of the numerical values shown in the row “No. 2” in FIG. 3, it is confirmed that the serotype is possibly Anatum (the candidates can be narrowed down).

In the case where it is previously known that the test microorganism is a Salmonella bacterium whose serotype belongs to the first group, if the mass-to-charge ratio corresponding to the marker protein YaiA is 7097, or if the combination of the mass-to-charge ratios of the 10 kinds of marker proteins is the combination of the numerical values shown in the row “No. 3” in FIG. 3, the serotype is identified as Newport. In the case where the test microorganism is a Salmonella bacterium with no information about its serotype, if the combination of the mass-to-charge ratios of the 10 kinds of marker proteins is the combination of the numerical values shown in the row “No. 3” in FIG. 3, it is confirmed that the serotype is possibly Newport (the candidates can be narrowed down).

In the case where it is previously known that the test microorganism is a Salmonella bacterium whose serotype belongs to the first group, if the mass-to-charge ratio corresponding to the marker protein S7 is 17460, or if the combination of the mass-to-charge ratios of the 10 kinds of marker proteins is the combination of the numerical values shown in the row “No. 4” in FIG. 3, the serotype is identified as Poona. In the case where the test microorganism is a Salmonella bacterium with no information about its serotype, if the combination of the mass-to-charge ratios of the 10 kinds of marker proteins is the combination of the numerical values shown in the row “No. 4” in FIG. 3, it is confirmed that the serotype is possibly Poona (the candidates can be narrowed down).

In the case where it is previously known that the test microorganism is a Salmonella bacterium whose serotype belongs to the first group, if the mass-to-charge ratio corresponding to the marker protein Gns is 6483 and the mass-to-charge ratio corresponding to YibT is 7894, or if the combination of the mass-to-charge ratios of the 10 kinds of marker proteins is the combination of the numerical values shown in the row “No. 5” in FIG. 3, the serotype is identified as Tallahassee. In the case where the test microorganism is a Salmonella bacterium with no information about its serotype, if the combination of the mass-to-charge ratios of the 10 kinds of marker proteins is the combination of the numerical values shown in the row “No. 5” in FIG. 3, it is confirmed that the serotype is possibly Tallahassee (the candidates can be narrowed down).

In an analysis of a bacterium belonging to the first group, if the mass-to-charge ratio corresponding to the marker protein Gns is 6542, or if the mass-to-charge ratio corresponding to L25 is 10569, or if the combination of the mass-to-charge ratios of the 10 kinds of marker proteins is the combination of the numerical values shown in the row “No. 6” in FIG. 3, the serotype is identified as Vellore. In the case where the test microorganism is a Salmonella bacterium with no information about its serotype, if the mass-to-charge ratio corresponding to the marker protein Gns is 6542, or if the mass-to-charge ratio corresponding to L25 is 10569, or if the combination of the mass-to-charge ratios of the 10 kinds of marker proteins is the combination of the numerical values shown in the row “No. 6” in FIG. 3, it is confirmed that the serotype is possibly Vellore (the candidates can be narrowed down).

Thus, under the condition that the test microorganism is a Salmonella bacterium, if it is previously known that its serotype belongs to either the first group or second group or to the first group, or if it is unknown whether or not the serotype is one of the serotypes belonging to the first and second groups, it is still possible to identify the serotype, or narrow down the candidates to fewer serotypes, based on the mass-to-charge ratio of each peak originating from one or more marker proteins selected from the group consisting of the 10 kinds of marker proteins and any combination of those marker proteins.

### MODES OF INVENTION

A person skilled in the art can understand that the previously described illustrative embodiments are specific examples of the following modes.

(Clause 1) A method for analyzing a microorganism according to one mode includes:

a step of reading, from a mass spectrum obtained by a mass spectrometric analysis of a sample containing a microorganism, a mass-to-charge ratio or ratios m/z of a peak or peaks originating from 1-10 kinds of proteins selected from the group, consisting of 10 kinds of proteins of gns, YaiA, YibT, PPI, L25, L21, S8, L17, L15 and S7 as well as any combination of the 10 kinds of proteins; and

an identification step configured to determine, based on the read mass-to-charge ratio or ratios, m/z, of the peak or peaks, whether or not the sample contains at least one of the following six serotypes of Salmonella bacteria: Abaetetuba, Anatum, Newport, Poona, Tallahassee and Vellore.

By the method for analyzing a microorganism described in Clause 1, it is possible to easily determine that a microorganism contained in a sample is one of the six predetermined serotypes of Salmonella bacteria which have conventionally been difficult to be distinguished from other serotypes of Salmonella.

(Clause 2) In the method for analyzing a microorganism described in Clause 1, the previously described identification step is replaced by:

an identification step configured to determine, based on the read mass-to-charge ratio or ratios, m/z, of the peak or peaks, whether or not the sample contains at least one of the following six serotypes of Salmonella bacteria: Abaetetuba, Anatum, Newport, Poona, Tallahassee and Vellore, or whether or not the sample contains at least one of the following 22 serotypes of Salmonella bacteria: Enteritidis, Typhimurium, Minnesota, Infantis, Brandenburg, Rissen, Schwarzengrund, Montevideo, Mbandaka, Orion, Pullorum_Gallinarum, Abony, Choleraesuis, Saintpaul, Braenderup, Pakistan, Thompson, Altona, Istanbul, Manhattan, Senftenberg, and Amsterdam.

By the method for analyzing a microorganism described in Clause 2, it is possible to easily determine that a microorganism contained in a sample is one of the six predetermined serotypes of Salmonella, or one of the 22 predetermined serotypes of Salmonella.

(Clause 3) In the method for analyzing a microorganism described in Clause 1 or 2, the method further includes a step of determining which of the six serotypes is contained in the sample, based on the mass-to-charge ratio or ratios m/z of a peak or peaks originating from 1-10 kinds of proteins selected from the group, consisting of the 10 kinds of proteins as well as any combination of the 10 kinds of proteins, when it is determined in the identification step that at least one of the six serotypes is contained in the sample.

By the method for analyzing a microorganism described in Clause 3, it is possible to determine which of the six serotypes of Salmonella bacteria is the microorganism contained in the sample.

(Clause 4) In the method for analyzing a microorganism described in Clause 1, the previously described identification step is replaced by:

a step of determining which of the six aforementioned serotypes is contained in the sample based on the read mass-to-charge ratio or ratios, m/z, of the peak or peaks.

By the method for analyzing a microorganism described in Clause 4, it is possible to determine which of the six predetermined serotypes of Salmonella bacteria that have conventionally been difficult to be distinguished from other serotypes of Salmonella is the microorganism contained in the sample.

(Clause 5) A program according to another mode is configured to execute the steps described in one of Clauses 1-4.

1. A method for analyzing a microorganism, comprising:
a step of reading, from a mass spectrum obtained by a mass spectrometric analysis of a sample containing a microorganism, a mass-to-charge ratio or ratios m/z of a peak or peaks originating from 1-10 kinds of proteins selected from the group, consisting of 10 kinds of proteins of gns, YaiA, YibT, PPI, L25, L21, S8, L17, L15 and S7 as well as any combination of the 10 kinds of proteins; and
an identification step configured to determine, based on the read mass-to-charge ratio or ratios, m/z, of the peak or peaks, whether or not the sample contains at least one of following six serotypes of Salmonella bacteria: Abaetetuba, Anatum, Newport, Poona, Tallahassee and Vellore.

2. A method for analyzing a microorganism, comprising, in place of the identification step in the method for analyzing a microorganism according to claim 1, an identification step configured to determine, based on the read mass-to-charge ratio or ratios, m/z, of the peak or peaks, whether or not the sample contains at least one of the following six serotypes of Salmonella bacteria: Abaetetuba, Anatum, Newport, Poona, Tallahassee and Vellore, or whether or not the sample contains at least one of the following 22 serotypes of Salmonella bacteria: Enteritidis, Typhimurium, Minnesota, Infantis, Brandenburg, Rissen, Schwarzengrund, Montevideo, Mbandaka, Orion, Pullorum_Gallinarum, Abony, Choleraesuis, Saintpaul, Braenderup, Pakistan, Thompson, Altona, Istanbul, Manhattan, Senftenberg, and Amsterdam.

3. The method for analyzing a microorganism according to claim 1, further comprising a step of determining which of the six serotypes is contained in the sample, based on the mass-to-charge ratio or ratios, m/z, of a peak or peaks originating from the 1-10 kinds of proteins selected from the group, consisting of the 10 kinds of proteins as well as any combination of the 10 kinds of proteins, when it is determined in the identification step that at least one of the six serotypes is contained in the sample.

4. The method for analyzing a microorganism according to claim 2, further comprising a step of determining which of the six serotypes is contained in the sample, based on the mass-to-charge ratio or ratios, m/z, of a peak or peaks originating from the 1-10 kinds of proteins selected from the group, consisting of the 10 kinds of proteins as well as any combination of the 10 kinds of proteins, when it is determined in the identification step that at least one of the six serotypes is contained in the sample.

5. A method for analyzing a microorganism, comprising, in place of the identification step in the method for analyzing a microorganism according to claim 1, a step of determining which of the six aforementioned serotypes is contained in the sample based on the read mass-to-charge ratio or ratios, m/z, of the peak or peaks.

6. A non-transitory computer readable medium recording a program for making a computer execute the steps described in claim 1.

7. A non-transitory computer readable medium recording a program for making a computer execute the steps described in claim 2.
